(19) United States
(12) Patent Application Publication
(10) Pub. No.: US 2012/0129787 A1
(43) Pub. Date: May 24, 2012

(54) Modified oligopeptides with anticancer properties and methods of obtaining them

(76) Inventors: Artur Martynov, Kharkov (UA); Boris S. Farber, Brooklyn, NY (US); Sonya Sophya Farber, New York, NY (US)

(21) Appl. No.: 12/931,458

(22) Filed: Feb. 1, 2011

Related U.S. Application Data
(63) Continuation of application No. PCT/RU2010/000690, filed on Nov. 22, 2010.

(51) Int. Cl. A61K 38/02; A61P 35/00

(52) U.S. Cl. 514/19.3

(57) ABSTRACT

This invention may be used in human and veterinary medicine for the creation of a drug that is effective perorally in the treatment of oncological illnesses.

Modified oligopeptides with anticancer properties distinct in that in the capacity of peptides, a mixture (assembly) of oligopeptides are used that are products of the hydrolysis of proteins with molecules changed to the opposite charge. This quantity of modified oligopeptides is capable of slowing down the activity of the heterodimer of b-importin cells and the replication of viruses whose replication cycle depends on the function of the nucleus, as well as selectively stopping the division of cancer cells and causing their death.

An assembly of modified oligopeptides based on a quasi-life, dynamic, self-organizing system that is effective in the treatment of oncological illnesses at all stages of the development of the cancer process where other drugs are ineffective. This drug has a wide spectrum of activity and a low level of toxicity; it is accessible for industrial production and effective at any stage of cancer.

### TECHNICAL FIELD

This invention is related to medicine—specifically, to oncology—and is intended for the treatment of oncologic diseases in humans.

### PREVIOUS LEVEL OF TECHNOLOGY

A high morbidity and mortality rate from malignant neoplasms requires changes in approaches to both diagnosis and treatment of this illness. Official methods of treatment of oncological illnesses are the most widely used: operations, radiation therapy, chemotherapy [¹]; the following began to be used not long ago: immune therapy [²], photodynamic therapy [³]. Recently, in medical circles, there has been a discussion of the issue of the non-correspondence of the practical effectiveness of modern cancer treatment methods with the theoretical calculations and biochemical models of the behavior of cancer cells under the influence of various damaging factors. This non-correspondence is related to: the inexplicability of the resistance of cancer to radiation therapy [⁴], cancer cells' resistance to chemotherapy even with the use of P-glycoprotein blockers [⁵], the non-effectiveness of bodies' immune reactions against tumors in the presence of a powerful immune response and a huge number of cancer antibodies in a patient's blood [⁶] and so on.

The data accumulated recently on the characteristics of tumor growth and the interaction of tumors with the macro-organism's systems may explain certain reactions of tumors to the influence of treatment.

In this application, we intend to publish the most up-to-date scientific data on cancerous tumors with the aim of developing a new strategy in the battle against this group of diseases.

Modern polychemotherapy methods are based on the application of a group of drugs that block various phases of the cell cycle simultaneously [⁷]. As a rule, this is a combination of anti-metabolics with bischlorethylamines, including drugs with platinum: Cisplatinum, Platidiam, and Platinol [⁸]. The simultaneous application of several similar drugs with various targets (phases of the cell cycle) might have led to the full regression of tumors due to the death of cancer cells in the division stage. However, later, Vakhtin demonstrated in his clonal concept of tumor growth that among the clones of fast-dividing cancer cells were found clones of dormant cells that had a low level of differentiation but did not divide at all [⁹]. It was also these cells that were fully indifferent to polychemotherapy. After the death of the fast-dividing cells, expression and amplification of P-glycoprotein, which is a calcium-dependent membrane pump, took place in the latent clones [¹⁰]. This glycoprotein quickly cleansed the cancer cells of all chemotherapy drugs before they had time to even penetrate the nucleus. The latent clones also began to multiply, but they were all already resistant to all kinds of chemotherapy, including drugs from the Taxane group, which only affected the Golgi complex tubulin in the cell. The cancer cells from the metastases were the most aggressive. They began to express a series of “aggressiveness” genes: collagenase, trypsin, fibrolysine, and other protease genes. The latter were helping the cells penetrate through vessel walls and metastasize quickly [¹¹]. This is especially characteristic of melanoblastomas, seminomas, and sarcomas. The cells practically lost differentiation and specialization, which sent them out of control of the body's regulatory systems. Because these cells are characterized by slow growth and ability to metastasize quickly, and they have a huge quantity of P-glycoprotein in the cell membrane, chemotherapy was practically ineffective [10]. Recently, a series of directions have appeared in the development of chemotherapy drugs: the use of chemosensitizers to block P-glycoproteins [¹²], as well as proteolysis inhibitors for blocking metastasis [¹³,¹⁴]. However, use of these drugs also does not address the problem of the resistance to chemotherapy on the part of latent tumor clones with slow-dividing metastatic cells. As soon as chemotherapy was discontinued, these cells began to divide and metastasize again.

There are several possible solutions to this kind of situation: the creation of drugs that have the ability to cause the apoptosis of cancer cells through the inactivation of the protein-synthesizing apparatus [¹⁵]. They should also have vectorness (be able to selectively collect only in cancer tumors) and not block cell division in the bone marrow, epithelium, hepatocytes, hair follicles, and so on. These substances should be larger in size than the P-glycoprotein channel, and the blocking of protein synthesis by these drugs in the cell should correspondingly automatically block the synthesis of proteases that facilitate cancer metastasis.

As a rule, cancer metastasis begins after the tumor has attained an average size of 3 cm (for adenocarcinomas); also, in the beginning, single metastatic cells spread throughout the body through the circulatory system and later they deposit themselves in large quantities in the lymph nodes [¹⁶]. The presence of collagenase and trypsin in the corresponding cancer clones facilitates significant metastasis through “lysis” of cancer cells through blood vessel walls. Surgical tumor removal, even when metastasis is not ready, but when tumors are about 3 cm requires shunting of the tumor's proliferative blood vessel system, and the trauma to the blood vessel system will lead to an expanded spread of cancer cells throughout the entire body from the locus of the operational intervention [11]. As Chissov et al wrote in their monographs, a scheme was proposed under which it, was supposed that metastasis would be brought to a minimum: before and after surgical treatment, radiation treatment of the cancer tumor was conducted. There was hope that even the penetration of some of the cells into the blood vessel network would not lead to metastasis, as those cells would be dead [¹⁷]. However, the practical results of the use of this scheme demonstrated that metastasis had even sped up in certain cases. A combination of chemo- and radiation therapy before and after surgical intervention did not produce the expected result either.

Biomolecular research has indicated that this ineffectiveness is conditioned on two factors: secretion by mother cancer cells of chalones that slow the growth of daughter metastasis; the presence of a huge number of macrophages, monocytes, and killer lymphocytes in the live tumor, which slowed down and prevented the spread of metastatic cells [¹⁸,¹⁹]. Thus it may be supposed that in the radiation of a tumor, first and foremost the cells of the immune system died—macrophages, T-killers, and monocytes—and the tumor became completely invisible to the immune system and began to metastasize very quickly even before the operation. After the operation, the cloned metastatic cells, deprived of the growth-stalling signal from the central chelones, began to divide very quickly. Also, they remained invisible to the immune system due to the death of the central tumor's machrophages.

One of the possible solutions to this situation may be considered limitation of local treatment methods (operative and radiation) for tumors of more than 3 cm and the replacement of these methods with chemotherapies and the application of P-glycoprotein blockers and methods of specific and adoptive immunotherapy in various modifications.

The first attempts to treat cancer with the use of the patient's immune system itself were conducted at the beginning of the century by injecting cancer patients with the tuberculosis vaccine [²⁰]. As J. L. Old demonstrated, in certain cases, full regression of both the initial cancer and the metastases was observed. The author demonstrated that this was conditioned on the activation of the functions of the so-called tumor necrosis factor (TNF) [²¹]. These were the first cases of cancer being cured that were authentically registered in the history of the disease. Since then, the methods of immunology have changed significantly: recombinant lymphokines have appeared: interferons [²²], interleukines [²³], lectin immunoinductors [²⁴] and polysaccharides [²⁵], automatic methods of adoptive cancer immunotherapy with interleukines using Rosenberg's method [²⁶], implantation of embryonic tissues and auto-vaccination with the body's own cancer cells using Govallo's method [²⁷]. These methods were effective on certain types of tumors to such an extent that they were implemented in many clinics and are effectively used to this day. Also, many of the problems of cancer immunotherapy remained: the full invisibility of far-flung metastases and many tumors to the immune system and patients' immune systems' insensitivity to lymphokines. For example, taking interleukine-2 in large doses will lead to significant pathology of patients' internal organs: necrosis, autoimmune processes [²⁸]. Long-term metastases remain invisible to the immunity even after massive doses of lymphokines. This bears witness to the presence of many obstacles on the path to the implementation of a whole set of immune reactions. In certain cases, during immunotherapy, full regression of tumors with metastases occurred [²⁹].

As Frolov demonstrated in his monograph, the majority of viruses persist in various tissues in the human body after infection [³⁰]. As may be seen in the example, the most widespread groups of viruses—the Herpesviridae family—immune deficits caused by representatives of this group such as cytomegalovirus (CMV) and herpes simplex (types 1 and 2) correlate with the presence of cancer tumors in the patients studied. When these people's lymphocytes were studied further, it turned out that CMV was persisting specifically in the CD4+ and CD8+ T lymphocytes (the killers and the helpers), and that often this caused coloenteritis in leukemic patients [³¹]. Also, the lymphocytes remained normal, but the capability of blast transformation, phagocytosis, and the ability to react to IL-2 were significantly lowered, and in certain cases, the use of IL2 led to the activation of adenoviruses, CMV, and influenza viruses persisting in the lymphocytes [³²]. In the culture, the infected lymphocytes did not lyse the cancer cells, while the lymphocytes from a healthy body continued to phagocyte them, which in certain cases allowed them to create successful bone marrow transplant in the presence of CMV in the material [³³]. In cervical cancer cases, the following pattern was found in the women: almost all the cases of cervical cancer were accompanied by the persistence and frequent flares of HSV-2 [³⁴]. It is also well-known and verified that this virus is attracted to cervical and ovarian macrophages in women, and macrophages are the main barrier to the spread of newly occurring tumors and are the main representatives of the local immune system [21]. This bears witness to both the tangential role of viruses in the etiology of cancer and their direct role in the immune system's indifference to tumors that have already occurred in the immunotherapy process.

Based on the facts listed above, it may be supposed that one of the effective directions in the raising of the activity of immune reaction to cancer may be considered the cleansing of the T lymphocytes and macrophages of persistent viruses, in part, of Herpesviridae family viruses as the most widespread among humans. At present, a certain etiological role of persistent influenza, paraflu, measles, and herpes viruses, as well as adenoviruses and papovaviruses, in carcinogene-sis, is well-known and -documented. Although there are not any drugs that will clear a patient's system of persistent viruses, we hope that soon both natural mechanisms of cleansing the genome of viral transposons and new drugs connected with these mechanisms will be discovered.

One of the new directions in the quick blockage of viral replication and the stoppage of the synthesis of viral proteins without the death of the cells themselves is the application of a new type of drug with antiviral activity: dynamic, quasi-life systems capable of adapting to a specific organism and specific viruses. This system is a composition of a thousand low-molecular oligopeptides with changed charges that are a dynamic pharmacophore with an imprecise structure.

The most similar prototype of a substance that is being patented is modified proteins and their use for the control of viral infections [35]. These are proteins that have been treated with various anhydrides and acylated substances: albumins, lactoferrin, transferrin, and lactalbumin The authors have also patented the mechanism of action for these proteins: a slowing of viral adhesion. These proteins must have a molecular mass of more than 60000 with a small amount of variance. The significant preventive anti-viral activity of these proteins has been demonstrated in experiments on cell cultures. The drugs demonstrated activity against HIV (human and Old World monkey), influenza, cytomegalovirus, the polio virus, the Semliki forest virus, the Sendai virus, the paraflu, and the Coxsackie virus. The authors have proven that acylated proteins are non-toxic and may protect animals from viral infection.

The prototype has some shortcomings: it is strictly a preventive drug (these proteins did not demonstrate a therapeutic effect on cells already infected by viruses) and do not have therapeutic properties for infected animals. In connection with the fact that the prototype is a high-molecular protein, it may only be taken parenterally; the drug is an individual combination and does not demonstrate the production of dynamic, self-organizing systems, and correspondingly, the viruses will quickly adapt to the drug. Also, the drug did not show anti-cancerous activity or the ability to rehabilitate the immune systems of cancer patients.

### DISCLOSURE OF THE INVENTION

At the basis of the invention, the task of synthesizing a mixture (assembly) of modified oligopeptides with anti-cancer properties and with a new mechanism of action whose use will allow a significant increase in the effectiveness of the treatment and reduce the treatment times of cancer patients, as well as to prevent the metastasis of immuno-contrasting tumors.

The task set is addressed through the synthesis of a mixture (assembly) of chemically modified peptides with anti-cancer properties, which is distinct in that first a partial hydrolysis of protein-containing raw material is conducted, and then a process of chemical modification of the quantity of oligopeptides obtained with a change in the charge to the molecules is conducted.

For synthesis, proteins may be used such as: ovalbumin (OA), human seralbumin (HSA), bovine seralbumin (BSA), a mixture of milk proteins (MMP), rabbit seralbumin (RSA), lysozyme (LZ), lactoalbumin (LA), casein (CS), soy protein (SP), a mixture thereof, milk (M), and whole egg white (WEW). For the purposes of enzymatic hydrolysis, pepsin, trypsin, chymotrypsin, papainase, K-proteinase, clostripain, thrombin, thermolysine, and elastase are used. The modifiers presented in FIG. 1 maybe used as modifying agents. In the experiment, the effectiveness of the drug on various in vivo and in ovo models was demonstrated, as described below. The synthesized oligopeptides are capable of slowing the activity of the heterodimers, of the a-b-importins of the cell, which transport viral polynucleotides from the cytoplasm to the nucleus. Accordingly, the slowing of these transport proteins will lead to the blockage of viruses whose replication depends on cell nucleus functions; also, the synthesis of proteins in cancer cells selectively stops. In addition, the drug is effective when taken orally.

The authors used an assembly of oligopeptides that were the product of the hydrolysis of polynucleotides, but the molecules' charges were changed to the opposite. “Assembly” is a term from supramolecular chemistry. The objects of supramolecular chemistry are supramolecular assemblies that self-assemble out of their complements—that is, fragments that have geometrical and chemical correspondence—similar to the self-assembly of the most complex three-dimensional structures in a live cell [³⁶,³⁷]

### SHORT DESCRIPTION OF DRAWINGS

FIG. 1—Structures of Chemical Modifiers Applied to Change the Charges of Modified Peptide (MP) Oligopeptide Molecules

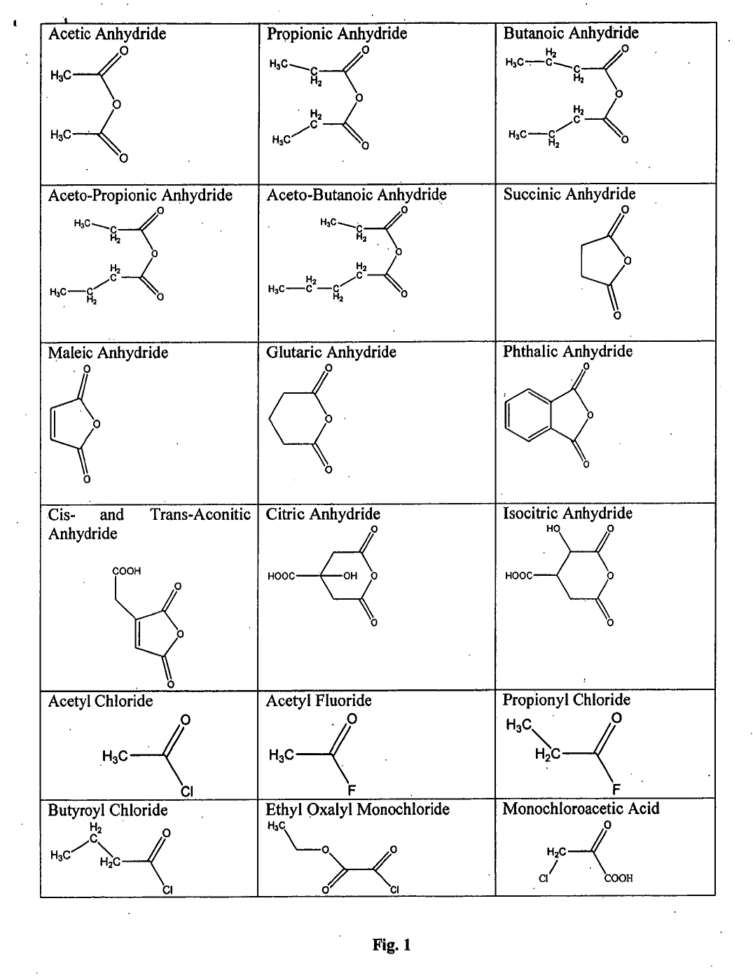

FIG. 2—Sarcoma. Hematoxylin-Eosin Dye

### BEST INVENTION IMPLEMENTATION OPTION

### EXAMPLE 1

Obtaining mixtures of modified peptides (MPs) with anti-viral properties that are capable of self-organizing into importins. Under aseptic conditions, 500 mg of ovalbumin is dissolved in 50 ml of distilled water and the pH is brought to 8.0 using 1 M of a sodium hydroxide solution. Trypsin is added, the solution is allowed to sit for 3-45 hours, and the hydrolysis of the ovalbumin with the formation of a peptide mixture is observed. To this mixture, 501-2000 mg of succinic anhydride. are mixed for 20 minutes at a temperature of 16-65° C. The mixture is run through membrane filters with the goal of sterilization, and is then poured into glass flagons.

### EXAMPLE 2

The anti-cancer activity of MPs. The determination of the anti-cancer activity of Anticanum in a cell culture was made in a culture of HeLa-2 cells. For this purpose, 20-120 mcg of MPs per nil of medium were added to the 199 medium. (See Table 2.) A culture without MPs in it was used as a control. Cultures were observed daily over the course of five days. The Minimum Active Dose (MAD) of Anticanum was also considered to be the minimum amount of the drug that caused a degeneration of 90-95% of the cells (Table 2).

TABLE 2
Comparative Sensitivity Characteristics of Cultures
of HeLa-2 Tumor Cells to Anticanum

| | | Anti-Cancerous Activity of | |
|---|---|---|---|
| | MAD in | MPs in Cell Culture | |
| Drug | mcg/ml | Control | Experiment |
| MP | 120 | 0 | ++++ |
| Taxotere | 10 | 0 | ++ |
| Physical | — | 0 | 0 |
| Solution | | | |

¹ Cytopathic activity;
++++ degeneration of 100% of the cells
0 lack of degeneration.

In establishing the minimum concentration of MPs that will slow the growth of cells, a comparison was made between the number of surviving cells and the concentration of MPs in the solution.

TABLE 3
The Effect of MPs on HeLa Cells

| | | Number of | | | |
|---|---|---|---|---|---|
| | | live cells after | | | |
| | Number of cells | incubation, | | Number of live cells | |
| Dose, | before incubation, | millions, ±1000 | | after incubation, % | |
| mcg/ml | millions | MP | Taxotere | MP | Taxotere |
| 20 | 151000 ± 1000 | 70000 | 140000 | 46 | 93 |
| 40 | 152000 ± 1000 | 37200 | 138000 | 24 | 91 |
| 60 | 151000 ± 1200 | 162000 | 1000 | 14 | 0.12 |
| 80 | 154000 ± 1000 | 0 | 127000 | 0 | 82 |
| 100 | 152000 ± 1000 | 0 | 140000 | 0 | 92 |
| 120 | 150000 ± 1000 | 0 | 152000 | 0 | 101 |

As may be seen in Table 3, an effective dose of MPs is between 80-120 mcg/ml solution.

MPs led to a 100% degeneration of tumor cells at a dose of 80-120 mcg/ml. To confirm the in vivo anti-tumor activity, MPs were studied in models of benzidine skin sarcoma and reinjected ascites adenocarcinomas in Barbados mice. In five mice with adenocarcinomas, the distribution of the MP liposome throughout the animals' bodies was also studied using a fluorescent probe (FTIC).

### EXAMPLE 4

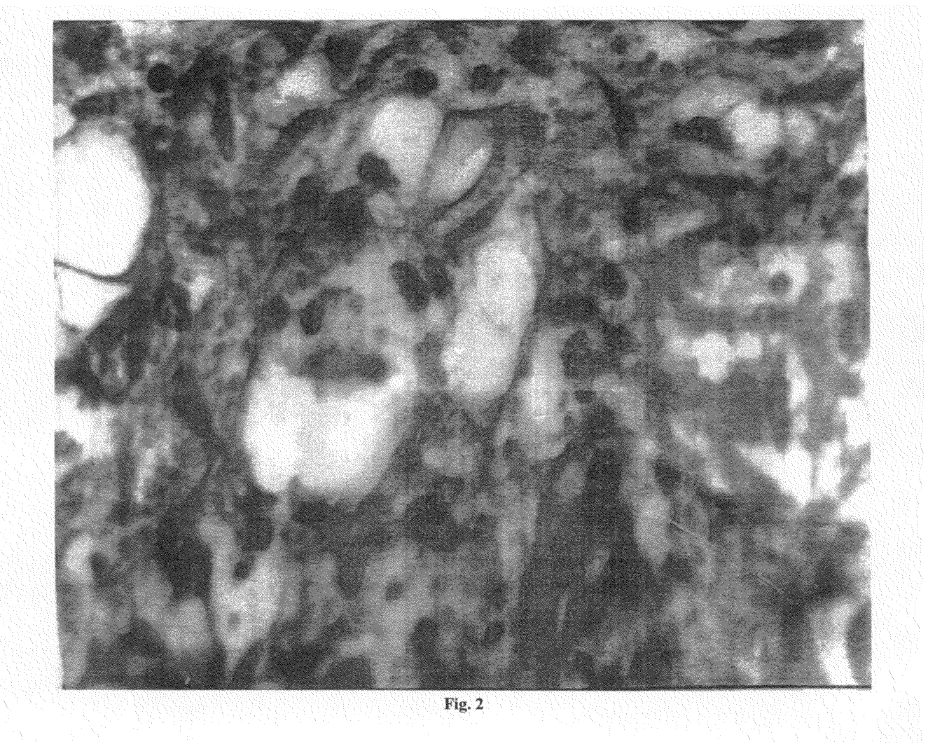

Study of the Anti-Cancer Activity of Anticanum on Benzidine Sarcoma. Before applying it to the silica gel, 7 ml of a solution of 2% benzidine and 0.9% sodium chloride was added until an opalescent suspension was formed (1 g silica gel for 5 ml NaCl solution). Twenty-five Barbados mice of both sexes with a weight of 18-20 g that were kept on a vivarium diet were administered benzidine and phorbol acetate immobilized on silica gel subcutaneously near the neck every three days. After two weeks, 18 animals had developed tumors of different sizes in the form of a small bump on the neck near the silica gel granulomas. The cytology of the tumors is presented in FIG. 2. Each group of animals was administered the corresponding compound parenterally at a dose of 100 mcg/kg weight twice a day for two weeks, beginning at 16 days after administration of the carcinogen.

TABLE 4
A Comparison of the Anti-Tumor Activity of MPs in Comparison to the
Combination of an Analog (Taxotere) and a Placebo (Physical Solution).

| | Weight of Animal (g) | |
|---|---|---|
| Drug Name | Before Treatment | After Treatment |
| Taxotere | 27 ± 2 | 23 ± 1.1 (2 mice died) |
| MP | 26 ± 2 | 15 ± 1.5 |
| Physical Solution | 26 ± 2 | 38 ± 1.3 (5 mice died) |

Note: n = 7, p > 0.05 in comparison with the control and previous data.

As may be seen in Table 4, MPs decreased the weight of the experimental animals by 11 g; the control animals' weight continued to increase, and some of them died. After the dissection of the silica gel granulomas, it was established that the animals treated with the MPs did not show signs that the granulomas had turned into malignant sarcomas.

The animals' survival rates are presented in Table 5.

TABLE 5
Survival Rates of Animals with Benzidine Skin Sarcoma

| Drug Name | Animal Survival, Days |
|---|---|
| Taxotere | 28 ± 1.1 |
| MP | 180 ± 5 |
| Physical Solution | 17 ± 0.9 |

Note: n = 10, p > 0.05 in comparison with the control and previous data.

Thus the MPs prolong the life of animals more than ten times as long as does Taxotere.

### EXAMPLE 5

A Study of the Anti-Tumor Activity of MPs on Ehrlich's Ascites Adenocarcinoma. The anti-tumor activity of the compositions were studied in models of Ehrlich's ascites carcinoma in young Barbados mice of both sexes with weights between 15-17 g (70 individuals), which were kept on a vivarium diet.

50 mice were inoculated from a mouse with adenocarcinoma using an insulin syringe with 0.1 ml ascitic fluid in the region of the liver. Within seven days, 4 mice showed signs of tumors (the body weight and belly size increased); three mice died on the second day; one mouse did not show signs of a tumor.

15 mice were administered MPs intraperitoneally (see Table 6). 15 more mice were administered the MPs intravenously, and fifteen more were administered a 0.9% solution of sodium chloride.

TABLE 6
Quantitative Biological and Statistical Characteristics in the Study of MP
Anti-Tumor Activity

| | | Time of Death of Animals | |
|---|---|---|---|
| | | after the First Injection | |
| | Form of | Average Value Days | |
| Substance | Introduction | Experimental Animals | Control Animals |
| MP | IV | 48 ± 7 | 5 ± 2 |
| -//- | IP | 52 ± 8 | 5 ± 2 |
| Taxotere | IV | 15 ± 1 | 3 ± 1 |
| -//- | IP | 14 ± 1 | 3 ± 1 |

Note: n = 10, p > 0.05 in comparison with the control and previous data.

MPs were given to those mice from which blood was drawn. Mice with Ehrlich's adenocarcinoma, after being given the tumor and treated, lived for 48-52 days when administered the modified substance, which is an average of 10 times longer than the control. At an accuracy level of more than 99.5%, we can confirm a significant increase in anticancer activity in MPs over the control, Taxotere. After dissection of the animals, signs of tumors and metastasis were not found in their bodies.

### EXAMPLE 6

A Study of the Distribution of MPs throughout the Body of Animals_For the study of the distribution of liposomes throughout animals' organs, MPs marked with FITC were used (after the enzymatic hydrolysis, 0.01% FITC was added to the peptide solutions; then it was acylated). The most fluorescence was observed in the ascitic fluids, which confirms the MPs' affinity for the tumor. In the control group, the MP distributed itself into the spleen, liver, and lymph nodes.

The distribution of the MPs was studied in the bodies of mice with tumors (five animals) and healthy rats (five animals) after a fluid administration of MP-FTIC. MP-FTIC was administered at a dosage of 240 mcg/kg. After five hours, the mice were euthanized and the fluorescence intensity was studied using an HP-F-40M fluorometer.

TABLE 7
Accumulation of MPs Dependent on Speed of Introduction

| | | Form of Anticanum | |
|---|---|---|---|
| Animals | Organ | Introduction | Fluorescence, % * |
| healthy rats | | slow infusion | |
| -∥- | plasma | | 43.5 ± 0.5 |
| -∥- | liver | | 12.5 ± 0.5 |
| -∥- | lungs | | 5.7 ± 0.5 |
| -∥- | spleen | | 5.0 ± 0.5 |
| -∥- | kidneys | | 32.2 ± 0.5 |
| -∥- | blood | | 11.5 ± 0.5 |
| | fast intravenous introduction | | |
| -∥- | plasma | | 40 ± 0.5 |
| -∥- | liver | | 58 ± 0.5 |
| -∥- | lungs | | 0.5 ± 0.5 |
| -∥- | spleen | | 0.5 ± 0.5 |
| -∥- | kidneys | | 0.5 ± 0.5 |
| -∥- | blood | | 0.5 ± 0.5 |
| mice with adenocarcinoma | | slow infusion | |
| -∥- | plasma | | 15 ± 0.5 |
| -∥- | liver | | 22 ± 0.5 |
| -∥- | lungs | | 1 ± 0.5 |
| -∥- | spleen | | 8 ± 0.5 |
| -∥- | kidneys | | 2 ± 0.5 |
| -∥- | ascitic fluid | | 52 ± 0.5 |
| | fast intravenous introduction | | |
| -∥- | plasma | | 15 ± 0.5 |
| -∥- | liver | | 44 ± 0.5 |
| -∥- | lungs | | 1 ± 0.5 |
| -∥- | spleen | | 8 ± 0.5 |
| -∥- | kidneys | | 2 ± 0.5 |
| -∥- | ascitic fluid | | 30 ± 0.5 |

* relative fluorescence: the percentage of fluorescence relative to the intensity of the fluorescence of the cells from the same tissues in the animals that did not receive the fluorescent marked drug.

As can be seen in the table, when a tumor is present; the aggregation of MPs goes there, but in dependence on the intensity of the infusion. The faster the MPs are introduced, the more of them are collected in the liver. It is therefore rational to introduce MPs either in the form of a slow infusion or in the form of delayed-release rectal suppositories.

This invention is related to human and veterinary medicine—specifically to oncology—and may be used in the treatment of oncological infections in animals and humans.

### INDUSTRIAL APPLICABILITY

This invention is related to veterinary and human medicine—specifically, to virology—and may be used for the creation of new drugs on the basis of dynamic, self-adapting and self-organizing systems for the treatment of viral infections in animals and humans The drugs obtained in this manner are completely ecologically safe, biodegradable, and fully metabolized both in patients' bodies and in the environment; the technology required to make them is completely without waste, does not require new, unique equipment and original reagents, and may be produced at any pharmaceutical company with unified production lines.

### REFERENCES

1 Treshchalina E M, Sedakova L A, Firsova G A. Selection of models for screening natural substances for anti-tumor activity.//Antib. and chemotherapy.-1990.-vol. 35, No. 2.-pp. 26-29
2 Ogarkov V I, Dobkin A I, Ryzhova E V, Okulov V B, Kiselev O I. Interleukine-2 and cancer immunotherapy.//Vopr. Onkologii.-1989.-vol. 35, No. 2.-pp. 141-151.
3 Berlin K., Jain R., Richert C. Are porphyrin mixtures favorable photodynamic anticancer drugs? A model study with combinatorial libraries of tetraphenylporphyrins.//Biotechnol Bioeng.-1998.-Vol. 61,No. 2.-P. 107-118
4 Denecke J., Fiedler K., Hacker-Klom U., Molenkamp G, Jurgens H., Wolff J. E. Multiple drug-resistant C6 glioma cells cross-resistant to irradiation.//Anticancer Res.-1997.-Vol. 17, No. 6D.-P. 4531-4534
5 Hendrikse N. H., de Vries E. G., Eriks-Fluks L., et al. A new in vivo method to study P-glycoprotein transport in tumors and the blood-brain barrier.//Cancer Res.-1999.-Vol. 59.-P. 2411-2416.
6 Gorodilova V V, Boeva M N. The immunology of tumor growth.-Moscow: Medicine, 1983.-236.
7 Blokhin N N, Perevodchikova N I. Chemotherapy of tumor illnesses.-Moscow: Medicine, 1986.-303 pp.
8 Gergel D., Misik V., Ondrias K. Effect of cisplatin, carboplatin and stobadine on lipid peroxidation of kidney homogenate and phosphatidylcholine liposomes.//Physiol Res.-1992.-Vol. 41, N 2.-P. 129-134
9 Vakhtin Yu B, Pinchuk V G, Shvemberger I N, Butenko Z A. A clonal selection concept of tumor growth.-Kiev: Nauk. dumka, 1987.-215 pp.
10 Morassutti C., Scaggiante B., Xodo L. E., Dapas B., Paroni G., Tolazzi G., et al. Reduction of mdr1 gene amplification in human multidrug-resistant LoVo DX cell line is promoted by triple helix-forming oligonucleotides. Antisense Nucleic Acid Drug Dev. 1999;9:261-70.
11 Liotta T. Tumor invasion and metastases-role of the extracellular matrix//Cancer. res.-1986.-Vol. 46., N 1.-P. 1-7.
12 Tian H., Pan O. C. Modulation of multidrug resistance by three bisbenzyl-isoquinolines in comparison with verapamil.//Chung Kuo Yao Li Hsueh Pao.-1997.-Vol. 18.-P. 455-458.
13 Rogelj B., Popovic T., Ritonja A., Strukelj B., Brzin J. Chelidocystatin, a novel phytocystatin from Chelidonium majus.//Phytochemistry.-1998.-Vol. 49, No. 6.-P. 1645-1649
14 Zheng Y., Guo X., Lin E. T., Benet L. Z. Overlapping substrate specificities of cytochrome P450 3A and P-glycoprotein for a novel cysteine protease inhibitor.//Drug Metab. Dispos.-1998.-Vol. 26.-P. 360-366.
15 Bantel H, Engels I H, Voelter W, Schulze-Osthoff K, Wesselborg S. Mistletoe lectin activates caspase-8/FLICE independently of death receptor signaling and enhances anticancer drug-induced apoptosis.//Cancer Res.-1999.-Vol. 59.-P. 2083-2090.
16 Schirrmacher V. Cancer metastasis: experimental approaches, theoretical concepts and impacts for treatment strategies//Advances in cancer research. Acad. Press.-1985-Vol. 43.-P. 1-73
17 Chissov V I, Kisileva E S, Antoshechkina A G et al. Combined and complex treatments of patients with malignant tumors.-Moscow: Medicine, 1989.-559.
18 Lian F, Li Y, Bhuiyan M, Sarkar F H. p53-independent apoptosis induced by genistein in lung cancer cells.//Nutr. Cancer.-1999.-Vol. 33.-P. 125-131.
19 Mori S, Murakami-Mori K, Bonavida B. Oncostatin M (OM) promotes the growth of DU 145 human prostate cancer cells, but not PC-3 or LNCaP, through the signaling of the OM specific receptor.//Anticancer Res.-1999.-Vol. 19.-P. 1011-1005.
20 Lobashevskiy A L, Kochemasova Z N. A tumor-necrotizing factor: The anti-tumor activity of certain microbes and their components.//Uspekhi sovr. biologii.-1988.-vol. 105, No. 2.-pp. 217-230
21 Old J. L. Tumor necrosis factor (TNF).//Science.-1985.-Vol. 230, No. 4726.-P. 630-632
22 Arora T., Floyd-Smith G., Espy M. J., Jelinek D. F. Dissociation between IFN-alpha-induced anti-viral and growth signaling pathways.//J. Immunol-1999.-Vol. 162.-P. 3289-3297.
23 Fu J, Zheng J, Fang W, Wu B. Effect of interleukin-6 on the growth of human lung cancer cell lined//Chin. Med. J. (Engl)-1998.-Vol. 111.-P. 265-268.
24 Timoshenko A. V., Kayser K, Drings P, Kolb G, Havemann K, Gabius H. J. Modulation of lectin-triggered superoxide release from neutrophils of tumor patients with and without chemotherapy.//Anticancer Res.-1993.-Vol. 13.-P. 1789-1792.
25 Burger R. A., Torres A. R., Warren R. P., Caldwell V. D., Hughes B. G. Echinacea-induced cytokine production by human macrophages.//Int J Immunopharmacol.-1997.-Vol. 19.-P. 371-379.
26 Rosenberg S. A., Packard B. S., Aebersold P. M. Use of tumor-infiltrating lymphocytes and interleukin-2 in the immunotherapy of patients with metastatic melanoma.//New England Journal of Medicine.-1988.-Vol. 319, N. 25.-P. 1676-1680
27 Govallo V I. Cancer immunology.//-Moscow, “Knowledge”., 1987.
28 Kendall A. Smith. Interleukin-2: inception, impact and implication.//Science.-1988.-Vol. 240, No. 4856, P. 1169-1176.
29 Mazumder A. M., Grimm E. A., Zhang H. Z. et al. Lysis of fresh human solid tumors by autologous lymphocytes activated in vitro by lectins.//Cancer res.-1982.-Vol. 42.-P. 913-918.
30 Frolov A F. Persistence of viruses (Mechanisms and clinical-epidemiological aspects). Vinnitsiya, N. I. Pirogov Vinnitsiya Medical University Press.-1995.-233 pp.
31 Ohata J, Matsuoka M, Yamashita T, Tojo A, Tani K, Asano S. CD4/CD8 double-positive adult T cell leukemia with preceding cytomegaloviral gastroenterocolitis.//Int. J. Hematol.-1999.-Vol. 69.-P. 92-95.
32 Kline J. N., Hunninghake G. M., He B, Monick M. M., Hunninghake G. W. Synergistic activation of the human cytomegalovirus major immediate early promoter by prostaglandin E2 and cytokines.//Exp. Lung. Res.-1998.-Vol. 24, P. 3-14.
33 Goi K, Sugita K, Miyamoto N, et al. Cord blood stem cell transplantation for infantile acute lymphoblastic leukemia after primary cytomegalovirus infection.//Rinsho Ketsueki.-1997.-Vol. 38, P. 1229-1233.
34 Infectious viruses and carcinogenesis./V. I. Struka, ed.-Kiev: Nauk dumka, 1987.-255 pp.
35 Robert Walter Jansen, Dirk Klaas Fokke Meijer, Grietje Molema, Erik Desire Alice De Clercq, Rudi Wilfried Jan Pauwels, Dominique Schols Modified proteins and their use for controlling viral infection//A61K 3804; A61K 3816, U.S. Pat. No. 5,869,457, Reg. Sep. 2, 1999. Appl. Sep. 4, 1997
36 http://dic.academic.ru/dic.nsf/ruwiki/79240
37 Jean-Marie Lehn. Supramolecular Chemistry. Concepts and Perspectives-Weinheim; New York; Basel; Cambridge; Tokyo: VCH Verlagsgesellschaft mbH, 1995.-P. 103 (Chapter 7)

1. Modified oligopeptides with anticancer properties distinct in that in the capacity of peptides, a mixture (assembly) of oligopeptides are used that are products of the hydrolysis of proteins with molecules changed to the opposite charge.

2. Modified oligopeptides with anticancer properties according to claim 1, distinct in that the charge of the molecules are changed through the formation of an amino group as a result of an acylation reaction between di- and tri-carboxylic acids and the remains of lysine and histidines in the oligopeptide mixture with the creation of new carboxyl groups.

3. Modified oligopeptides with anticancer properties according to claim 1, distinct in that the charge of the molecules are changed through the formation of a substitution amino group as a result of an alkylation reaction between monochloroacetic acid and the remains of lysine and histidines in the oligopeptide mixture with the creation of new carboxyl groups.

4. A method of obtaining modified oligopeptides with anticancer properties distinct in that to obtain them, first a partial hydrolysis of protein-containing raw material is conducted, and then a process of chemical modification of the quantity of oligopeptides obtained with a change in the charge to the molecules is conducted; this is used as an anti-viral vehicle for the composition of the oligopeptides obtained.

5. A method of obtaining modified oligopeptides with anticancer properties according to claim 4, distinct in that as a protein-containing raw material, an individual protein is used.

6. A method of obtaining modified oligopeptides with anticancer properties according to claim 4, distinct in that as a protein-containing raw material, a mixture of proteins is used.

7. A method of obtaining modified oligopeptides with anticancer properties according to claim 4, distinct in that as a protein-containing raw material, milk is used.

8. A method of obtaining modified oligopeptides with anticancer properties according to claim 4, distinct in that as a protein-containing raw material, primary egg albumen is used.

9. A method of obtaining modified oligopeptides with anticancer properties according to claim 4, distinct in that for the partial hydrolysis of protein-containing raw material, enzymatic hydrolysis is used.

10. A method of obtaining modified oligopeptides with anticancer properties according to claim 4, distinct in that for the partial hydrolysis of protein-containing raw material, acidic hydrolysis is used.

11. A method of obtaining modified oligopeptides with anticancer properties according to claim 4, distinct in that for the partial hydrolysis of protein-containing raw material, alkaline hydrolysis is used.

12. A method of obtaining modified oligopeptides with anticancer properties according to claim 4, distinct in that for the partial hydrolysis of protein-containing raw material, synthetic peptidases are used.

13. A method of obtaining modified oligopeptides with anticancer properties according to claim 4, distinct in that for the chemical modification of an amount of oligopeptides, succinic anhydride is used.

14. A method of obtaining modified oligopeptides with anticancer properties according to claim 4, distinct in that for the chemical modification of an amount of oligopeptides, monochloroacetic acid is used.
